(19) United States
(12) Patent Application Publication
(10) Pub. No.: US 2010/0291179 A1
(43) Pub. Date: Nov. 18, 2010

(54) COMPOSITE BIOMATERIAL FOR CONTROLLED RELEASE OF ACTIVE INGREDIENTS

(75) Inventors: Dieter Stoll, Moessingen (DE); Reinout Stoop, Voorschoten (NL); Juliana Vaz Nürnberger, Stuttgart (DE); Christoph Gaissmaier, Tuebingon (DE); Michael Ahlers, Eberbach (DE); Claudia Bohnsack, Hamburg (DE)

Correspondence Address: KLARQUIST SPARKMAN, LLP, 121 SW SALMON STREET, SUITE 1600, PORTLAND, OR, 97204, US

(73) Assignee: NMI Naturwissenschaftliches und Medizinishes Institut an der Universitaet Tuebingen

(21) Appl. No.: 12/762,230

(22) Filed: Apr. 16, 2010

Related U.S. Application Data
(63) Continuation of application No. PCT/EP08/08801, filed on Oct. 17, 2008.

(30) Foreign Application Priority Data
Oct. 18, 2007 (DE) 10 2007 051 059.6

(51) Int. Cl. A61K 9/10; A61K 38/18; A61F 2/00; A61P 35/00; C12N 11/08

(52) U.S. Cl. 424/423; 424/484; 514/8.8; 424/485; 435/180

(57) ABSTRACT

The present invention relates to a composite biomaterial and a process for producing a composite biomaterial with controlled release of active ingredients, comprising a three-dimensional polymer-based support structure, and a polymer-based matrix structure. The active ingredients are in this case incorporated into the composite biomaterial via the matrix structure, and the composite biomaterial is obtainable by introducing the matrix structure into the support structure. The active ingredients may in this case be incorporated for example adhesively, via specific binder molecules or via enzyme-labile linker molecules via the matrix structure into the composite biomaterial.

### CROSS REFERENCES TO RELATED APPLICATIONS

This application is a continuation of international patent application PCT/EP2008/008801, filed on Oct. 17, 2008 designating the U.S., which international patent application has been published in German language and claims priority from German patent application DE 10 2007 051 059.6, filed on Oct. 18, 2007. The entire contents of these priority applications are incorporated herein by reference.

### BACKGROUND OF THE INVENTION

The present invention relates to a composite biomaterial with controlled release of active ingredients, comprising a three-dimensional polymer-based support structure, and a polymer-based matrix structure.

Composite biomaterials are required and employed in the state of the art for various purposes, such as, for example, in the area of so-called tissue engineering in regenerative therapy. The aim of this technique is to reconstruct, and make capable of functioning, human or animal tissue which is in any way injured, damaged, degenerated or the like, by absorbable implants. The intention with the implants, which usually both form a protective matrix for the newly forming tissue, and actively promote cell growth, is to achieve regeneration of affected tissue. For this regrowing of tissue, cells of an organism are either expanded outside the target tissue to be regenerated and, after the cultivation, implanted in the same or another organism for tissue neogenesis or regeneration. A further possibility is for active ingredients to be incorporated into the biomaterial and, after implantation of this biomaterial into the affected tissue, to be released into the tissue surrounding the biomaterial, by which means it is possible to influence cells which surround the biomaterial and/or migrate into the latter by the active ingredients, for example to cell growth.

Such implants or biomaterials are usually produced from a plurality of components and therefore form composite materials. They are employed for example in the regeneration of articular cartilage. Such biomaterials must therefore, besides biocompatible and bioactive properties, also have very good mechanical strength and stability in order to protect the cells contained in them and/or to be taken up, and in order to withstand in vivo forces occurring during a movement.

On the other hand, such support structures are also used as active ingredient-releasing biomaterials (drug-release materials) through which active ingredients incorporated in the support structure can be released specifically at the place where the support structure is implanted.

An important aspect in this connection for both uses of the biomaterials is that they are biodegradable and non-toxic, and exert no stimulating effect on cells involved in inflammations. In relation, practical handling and questions of the approval of such implants is also becoming increasingly important.

A substantial improvement in the biomaterials used to date for these applications is therefore also that they not only act as active ingredient supplier but also respond to cell signals or signal substances which are given off by the cells/cell assemblages surrounding them. It is further desirable for the biomaterials to be able, owing to substances present on or in them or because of their structure or composition, to influence or even control the differentiation, the growth and/or the metabolism of cells, for example through the release of appropriate substances from the biomaterial, such as, for example, growth factors. However, at the same time, this function exerted by the biomaterial is to be controllable with the biomaterial components to be employed. This means that the released substances respond in the best case to the in vivo release profiles of the factors which are produced during natural tissue morphogenesis or during tissue neogenesis.

It is further often desirable with biomaterials loaded with active ingredients to be released for the latter to be capable of controlled release, i.e. for example with a particular rate and with a particular speed, or for in fact two or more active ingredients to be capable of targeted release with identical or different rates.

At present, both natural and synthetic biomaterials are employed for the applications explained above in tissue engineering and as drug release implants, and this in a wide variety of forms, including fibrous structures, porous sponges, woven net structures and hydrogels.

It is known in the state of the art to bind growth factors directly to the biomaterial support structures. Thus, for example, Young et al. (J. Control Release 109 (2005) 256-274) showed direct binding of basic fibroblast growth factor (bFGF) to gelatine.

A composite biomaterial consisting of a crosslinked gelatine matrix and a collagenous support layer is described in WO 2007/057175.

The use of hydrogels or of microspheres is also known. Hydrogels are based on hydrophilic polymers which are crosslinked in order to avoid dissolution in water. Since hydrogels may contain large amounts of water, they can be employed for releasing protein drug substances. A variant of hydrogels is the formation of so-called microspheres which are loaded with, for example, growth factors and which are subsequently incorporated into other biomaterials.

Lutolf et al. (Proc. Natl. Acad. Sci. USA (2003) 100:5413-5418) generated a hydrogel by putting an integrin-binding peptide, and a peptide having a matrix metalloprotease-sensitive sequence into a solution containing PEG (polyethylene glycol)-tetravinyl sulphone. Bone morphogenetic protein 2 (BMP-2) was also added and was released in the subsequent experiments owing to the degradation of the hydrogels by MMPs.

In addition, WO 97/41899 A1 discloses gelatine gels with adhesively bound growth factors as wound dressing, and EP 0 988 108 B1 discloses the use of superabsorbent porous hydrogel composites.

Despite the numerous biomaterials known and described in the prior art for use in tissue engineering or as drug release implants, there is still an enormous need for improved biomaterials which can be employed flexibly.

### SUMMARY OF THE INVENTION

It is therefore an object of the present invention to provide a composite biomaterial which makes it possible for one or more active ingredients to be released from the biomaterial in a time-controlled and/or quantitatively controlled manner, and at the same time the production of the biomaterial is simple and provides a certain flexibility and advantages in production and approval.

This object is achieved in the present case by a composite biomaterial comprising a three-dimensional polymer-based support structure, and a polymer-based matrix structure, with the active ingredients being incorporated into the composite biomaterial via the matrix structure. In this connection, it is preferred in one embodiment for the composite biomaterial to be obtainable by introducing the matrix structure including the active ingredients into the support structure.

The object is further achieved by a process for producing a composite biomaterial comprising a three-dimensional polymer-based support structure, and a polymer-based matrix structure, which includes the following steps:
a) provision of i) polymer-based support material and ii) polymer-based matrix material, where appropriate with a bound active ingredient;
b) where appropriate hydrophilization of the support material; and
c) introduction of the active ingredient-doped matrix material into the hydrophilized support material from step b) to produce a composite biomaterial with a support structure and a matrix structure which is active ingredient-doped where appropriate.

The object underlying the invention is fully achieved in this way.

With the composite biomaterial of the invention there is provided a matrix which is a flexible starting material for numerous medical, biological and laboratory technical applications. The composite biomaterial of the invention has the advantage over biomaterials known in the prior art that it can be produced in a simple modular fashion, can be manufactured, analysed and stored separated into its components, and can be provided individually and specifically for the particular desired application.

“Composite biomaterial” means in the present connection any biological or synthetic material which has a biological functionality, is biodegradable, and includes at least two components. The composite biomaterial moreover displays overall adequate stability and flexibility in order both to be capable of simple handling and to be capable of taking over various biological functions, such as, for example, those of an implant, of a medicament-releasing pad, of a three-dimensional matrix for cell cultures, of a bioactive coating for implantable medical devices, etc.

“Support structure” means in the present connection any structure which has the function of a support for a further structure to be introduced into the support structure, i.e. which acts as support or framework for the further components to be introduced into this support structure.

“Matrix structure” means in the present case any material which acts as recipient structure or material for substances or entities, or any material to which substances or entities can be bound, whether adhesively, covalently, etc.

“Active ingredient” means in the present case any substance which exerts in any way an effect on cells, cell assemblages or cell constituents and can initiate reactions of the cell or cell constituents there. This effect may become evident for example through the active ingredient triggering a particular reaction in the cells which in turn itself has an effect on the cells.

The composite biomaterials of the invention can be employed for example in the area of tissue engineering explained at the outset, and there serve for example as replacement for cell-doped implants. The composite biomaterials are thus able after implantation thereof at a desired site of action for example to stimulate local differentiation and proliferation of stem cells from the surroundings by the growth factors bound on the biomaterials and to be released, and thus induce and control a desired tissue regeneration.

The composite biomaterials of the invention are further suitable for example as implants for treating degenerative joint diseases, in which case the advantage of the present invention compared with current therapeutic policies with systemic administration of active ingredients is that the treated patients are exposed to a distinctly lower dose of active ingredient, based on the total patients, owing to a local high release of active ingredient which specifically assists regeneration, and thus the risk of unwanted side effects or toxic effects can be greatly reduced. Especially with administration of growth factors, it is possible through the use of the composite biomaterials of the invention, with the temporally and spatially accurately dosable administration of active ingredient which is possible therewith, to prevent induction of tumour growth in other body regions for example.

The composite biomaterial can additionally be employed as three-dimensional matrix for cell culturing in research, for example to investigate certain properties of cells or to investigate the effects of particular active ingredients incorporated on/in the composite biomaterial on cells. The modular structure of the material is advantageous in this connection and makes high flexibility and diversity when employed in R&E applications possible.

According to one aspect of the invention, the support structure includes a polymer which is selected from the group of synthetic polymers (e.g. polylactide, polyglycolide, polyethylene glycol, polyvinyl alcohol, polyimine, polyurethane, polyhydroxybutyric acid, polyethylene terephthalate, polytetrafluoroethylene, polypropylene, etc.) or of biopolymers such as, for example, polypeptides/proteins (e.g. gelatine, collagen, fibrin, etc.) or polysaccharides (e.g. starch, cellulose, agarose, alginate, dextran, chitosan, hyaluronic acid, etc.).

This has the advantage that it is possible to use materials to construct the support structure which have already been tested in the area of regenerative therapy and are already employed as approved implants and have proved a suitable starting material in this area.

According to another aspect of the invention the matrix structure includes a polymer which is selected from synthetic polymers (e.g. polyimine, polyvinyl alcohol, polyethylene glycol, polyurethane, polyaspartic acid, polylysine, etc.) or biopolymers (gelatine, collagen fragments, albumin, dextran, heparan sulphate, hyaluronic acid, chitosan, nucleic acids, peptides, lipids, etc.) or polymeric molecules produced biotechnologically (e.g. aptamers, fusion proteins, etc.) which make incorporation into the support structure and a chemical or physical attachment of active ingredients possible. The polymers of the matrix structure may moreover in turn display different properties and also be employed as mixture.

According to one aspect of the invention, gelatine can, owing to the starting materials used for the production (pig skin, ox hide, bone) or owing to the manufacturing conditions (alkaline, acidic), be produced with different chemical and physicochemical properties and be optimally selected by further fractionation steps in relation to molecular weight, isoelectric point or hydrophobicity for the intended task as matrix component. It is possible to employ for this purpose well-known screening systems such as, for example, surface plasmon resonance spectroscopy (“Biacore Technology”) which make it possible to measure comparatively the physicochemical interaction between matrix component and active ingredient or support component.

According to another aspect of the invention, the active is selected from at least one of the groups of low molecular weight active ingredients (anti-inflammatory agents, enzyme inhibitors, antibiotics, hormones, etc.), growth factors (e.g. bone morphogenetic proteins BMP-2, -7, etc.), or other protein active ingredients (for example chemokines etc.). The active ingredients also include bioactive peptides such as, for example, type I collagen peptides with an average molecular weight of ≦3500 D, for which a stimulating effect on chondrocytes has been demonstrated.

According to one aspect of the invention, the active ingredient to be incorporated into the biomaterial by a nonspecific binding to the matrix structure.

The introduction of the active ingredient and the binding to the matrix structure can take place optimally through the use of mediators—components which both efficiently solubilize the active ingredient and exhibit a high compatibility with the matrix. In the case of insoluble gelatine matrices, soluble low molecular weight gelatine fragments are suitable for this purpose.

This has the advantage that the active ingredient to be introduced is bound to the matrix structure under very mild conditions by physicochemical interactions merely by simply mixing with the matrix structure and is taken up together with the matrix structure into the support structure. The active ingredient thus present in the composite biomaterial is therefore not modified and can, for example when employed for the purpose of tissue regeneration, be released simply from the biomaterial after implantation without the need for the active ingredient to be actively dissolved out of the biomaterial by further measures. The control of active ingredient release takes place in this case 1. via the equilibrium constant of the binding of active ingredient and matrix structure, which can be determined by simple interaction screening, and 2. through the diffusion effect in the support. A further advantage of this embodiment is the simple production procedure for the composite material which makes it possible for the method to be optimized quickly and production to take place favourably from the regulatory point of view.

According to another aspect, the active ingredient is bound to the matrix structure via binder molecules, in particular peptides, which specifically bind the active ingredient, and can be incorporated as active ingredient matrix into the biomaterial.

This has the advantage that the active ingredients can be introduced into the matrix structure and thus into the composite biomaterial specifically via binder molecules which specifically bind the active ingredients, in the preferred form in a stable, bioactive conformation. Use is made in this connection in particular of bindings already known in the state of the art between active ingredients and peptides, such as, for example, the binding of BMP-2 (bone morphogenetic protein 2) to certain cyclopeptides (Behnam, K., Phillips, M. L., Silva, J. D., Brochmann, E. J., Duarte, M. E., and Murray, S. S. (2005) BMP binding peptide: a BMP-2 enhancing factor deduced from the sequence of native bovine bone morphogenetic protein/non-collagenous protein. J. Orthop Res 23, 175-180).

It will be appreciated that it is possible to employ in this embodiment of the composite biomaterial any binder molecule which makes specific binding of active ingredients and their release, for example over time under physiological conditions, possible. The “binder molecule” may in this connection be for example a receptor, a cofactor or a protein of the extracellular matrix. It is additionally possible to generate specifically for this purpose also aptamers, antibodies, antibody fragments, affibodies and other binder molecules with suitable binding constant and affinity.

According to yet another aspect of the invention cyclopeptides are used which are bound covalently via linkers to the matrix structure. According to one aspect of the invention, the attachment of the linker takes place via reactions of the thiol reactants, such as, for example, in cystein, and maleimide, or N-hydroxylamine and aldehyde, amine-aldehyde, or other specific coupling methods which are known in the state of the art and which can be formed for example under physiological conditions.

With certain active ingredients it is possible for their activity to be increased further in this embodiment for example through binder molecules which themselves are biologically active as cofactors. In these cases, the biological activity of the active ingredient becomes lower on release from the matrix structure. Such composite materials make it possible advantageously to reduce the therapeutic amount of active ingredient and provide improved pharmacological safety because potential side effects are minimised thereby.

The inventors were able to show in their own experiments that BMP-2 which was bound via various cyclopeptides to the matrix structure and thus into the biomaterial retained its activity in the support, interacted with cells and was scarcely released into the surroundings of the support over a period of more than three weeks.

The invention therefore particularly relates to a composite biomaterial for controlled release and administration of active ingredients, comprising a three-dimensional polymer-based support structure, and a polymer-based matrix structure which is introduced into the three-dimensional support structure, where the active ingredient is bound to the matrix structure via peptides which specifically bind the active ingredients.

According to one aspect of the invention, the active ingredient is growth factor BMP-2 (bone morphogenic protein 2) which is specifically bound to the matrix structure via cyclopeptides specific for BMP-2. According to one aspect, the peptide specifically binding the active ingredient is derived from a protein which is selected from bovine fetuin, bovine BMP-binding protein (BBP) or the receptor for transforming growth factor β(TGFβ-2).

The inventors have been able to show in their own experiments that BMP-2 can be efficiently bound to the matrix structure with peptides having sequences of the proteins mentioned, and at the same time efficient release of the active ingredient into the surroundings was detectable over a prolonged period. Also, the inventors were able to show that the biological activity of the growth factor was in fact increased by the cyclopeptides.

According to another aspect of the invention, the peptide which specifically binds the active ingredient has a sequence which is selected from one of SEQ ID No. 1 to 12 from FIG. 4 or the appended sequence listing.

The listed peptides could stably be cyclized and immobilized via a spacer and a linker on the matrix structure, thus making it possible to investigate the interactions between immobilized cyclopeptide and BMP-2. It emerged from this that there was a marked concentration-dependent binding to and release of BMP-2 from the cyclopeptide.

According to yet another aspect of the invention, the active ingredient is covalently bound to the matrix structure, in particular via linker molecules which are enzymatically cleavable, in particular for example by a protease, glycanase, nuclease etc.

The invention therefore also relates to composite biomaterials for controlled release of active ingredients, comprising a three-dimensional polymer-based support structure, and a polymer-based matrix structure which is introduced into the three-dimensional support structure, where the active ingredient is bound to the matrix structure via linker molecules covalently bound to the matrix structure, where the linker molecules can be cleaved by enzymes, for example by proteases.

This measure advantageously makes use of the surroundings into which the composite biomaterial is introduced for example in the form of an implant, or the enzymatic circumstances of the surroundings, according to which the release of the active ingredients can be made dependent on the predicted or calculated activity for example of cell-intrinsic proteases.

According to yet another aspect of the invention, the peptides are cleavable by a disease-associated matrix metalloprotease MMP (e.g. MMP-1, 3, 7, 8, 9, 10, 13 etc). Matrix metalloproteinases are members of the large family of zinc-dependent endopeptidases which are substantially involved in degradation of the extracellular matrix. They are formed by a large number of cells and secreted as so-called zymogens. The activity of the MMPs secreted by the cells is then utilised to cleave the MMP-sensitive peptides via which the active ingredients are bound in the biomaterial, and thus to release the active ingredients.

The successful release of substances by an MMP-7 activity with various peptide substrates could be shown with the composite biomaterial according to the invention.

According to another aspect, the peptide bound to the matrix structure has a sequence which is selected from one of the SEQ ID No. 13 to 20 from FIG. 7 and the appended sequence listing.

As mentioned herein before, the invention also relates to a process for producing a composite biomaterial comprising a three-dimensional polymer-based support structure, and a polymer-based matrix structure, with the following steps:
a) provision of i) polymer-based support material and ii) polymer-based matrix material;
b) where appropriate hydrophilization of the support material; and
c) introduction of the matrix material into the hydrophilized support material from step b) to produce a composite biomaterial with a support structure and a matrix structure introduced therein.

This measure has the advantage that the support material and the matrix material can be prepared separately, thus making it possible for both materials, or else only one thereof, to be produced for storage and to be stored stably where appropriate under various advantageous conditions. It is advantageous in this connection that production processes of support material and matrix-active ingredient material take place separately and thus can be optimized separately. In relation to approval of the composite materials as medical device or pharmaceutical product, the individual processes can be validated and documented separately and any purification of the matrix-active ingredient materials which is necessary to remove chemicals from the production process can be ensured with established methods of pharmaceutical manufacture (e.g. chromatography). A corresponding purification cannot be carried out in three-dimensional support structures.

A further advantage is that for example the matrix material is modified specifically for the particular desired use before introduction into the support material, for example by doping the matrix material with active ingredient, i.e. by introducing the active ingredient into the matrix material by diffusion, also for example by modification with peptides which in turn specifically recognize and bind particular active ingredients, or else by protease-labile peptides and active ingredients bound thereto. This separate provision therefore also makes individual combination of the composite biomaterial possible. Accordingly, selection of the support material and of the matrix material can be adapted to various applications, just like the selection and preparation of selective binder molecules, or suitable protease substrates. In addition, when the active ingredients are bound via selective binding molecules, the dissociation constant can be determined to predict the kinetics of release, and in the case of binding via protease-labile peptides the duration of release can be made dependent on the measured or expected protease activity at the site of action.

It is also possible advantageously to incorporate a plurality of different active ingredients into the composite biomaterial, and even to adjust the respective kinetics of release individually. It is also possible where appropriate to add the active ingredient immediately before use of the composite biomaterial, so that premature degradation of the active ingredients and thus premature inactivity thereof is avoided.

It is particularly preferred in the process of the invention for all the support material and/or the matrix material to include a material as described hereinbefore for the composite biomaterial.

The gelatine supports used in this connection can be produced for example in accordance with WO 2005 111 121 A2.

According to another aspect of the invention, the active ingredient used in the process is selected from one of the group of low molecular weight active ingredients (anti-inflammatory agents, enzyme inhibitors, antibiotics, hormones etc.), growth factors (e.g. bone morphogenetic proteins BMP-2, -7, etc.), or other protein active ingredients (for example chemokines etc.). According to one aspect of the invention, the active ingredient is incorporated into the biomaterial by a nonspecific binding to the matrix structure.

Also, the present invention relates to a method for treating degenerated or diseased tissue of a patient, wherein a composite biomaterial according to the invention is administered or implanted to the patient in need thereof, in particular a composite biomaterial which is produced by the process of the invention.

It is thus advantageously possible to employ the composite biomaterial of the invention which is loaded with appropriate active ingredients, for example BMP-2, for treatment of joint damage and for regeneration of cartilage. It is possible through the local delivery, limited to the composite biomaterial, of appropriate active ingredients such as growth factors for cells or tissue which surrounds the implanted composite biomaterial, to be influenced in a targeted manner, such as, for example, stem cells to differentiate and proliferate and thus for neogenesis of tissue. The local release of growth factors in a support is particularly advantageous because high effective concentrations can be achieved thereby with very low systemic concentrations of the active ingredients. It is thus possible to avoid unwanted side effects which may consist for example of use of growth factors in induction of tumours or increasing tumour cell proliferation.

The composite biomaterial of the invention can, however, also be employed as medicament with which local chemotherapeutics are to be delivered to the tissue surrounding the composite biomaterial or to the cells. It is advantageous in this connection for the active ingredients incorporated on/in the composite biomaterial to be delivered to the surroundings by kinetics of release which are adjusted in a targeted manner, for example via a protease activity. For example, targeted and specific treatment of cancer cells is possible thereby, so that systemic administration of chemotherapeutics which are usually associated with many side effects and stresses for a patient's body are advantageously avoided.

The invention further relates to a method for cultivating cells in vitro, wherein a composite biomaterial of the invention is provided or a composite biomaterial which is prepared by the process of the invention, and wherein the composite material is seeded with a cell or a cell population and cultivated under suitable conditions that allow growth of the cells.

In this use therefore, the composite biomaterial is employed as three-dimensional matrix which is loaded with the particular desired active ingredients and onto which cells are seeded. It is possible thereby on the one hand to investigate the effects of particular active ingredients—alone or in combination with other active ingredients—on different cells or types of cells. One advantage of this composite biomaterial is the modular concept which makes rapid and efficient combination of different active ingredient-matrix combinations possible with different support structures, support porosities, etc.

Further advantages are evident from the figures and the following description.

It will be appreciated that the features mentioned above and to be explained hereinafter can be used not only in the combination indicated in each case, but also in other combinations or alone, without departing from the scope of the present invention.

### BRIEF DESCRIPTION OF THE DRAWINGS

The invention is explained further by means of the following figures and the examples. These show

FIG. 1 a diagrammatic, sectional depiction of the structure of the composite biomaterial of the invention in a first embodiment;

FIG. 2 a diagrammatic, sectional depiction of the structure of the composite biomaterial of the invention in a second embodiment in which the active ingredients are taken up into the biomaterial via specific binder molecules;

FIG. 3 a diagrammatic, sectional depiction of the structure of the composite biomaterial of the invention in a third embodiment in which the active ingredients are taken up into the biomaterial via specific, enzyme-labile linkers;

FIG. 4 the tabular listing of cyclic peptides used by way of example for specific BMP-2 binding according to the second embodiment.

FIG. 5 an overview of the peptide library employed to identify the protease sensitivity (MMP-7) of various peptide sequences,

FIG. 6 a tabular overview of the increments in the pseudo half-life of proteolysis (MMP-7) determined in investigations of the kinetics of degradation of the peptide libraries from FIG. 5; and

FIG. 7 a tabular overview of the comparison of the factors calculated from the increments in FIG. 6 for MMP-7 proteolysis of octapeptides with the experimentally determined pseudo half-lives of proteolysis.

### DETAILED DESCRIPTION OF THE FIGURES

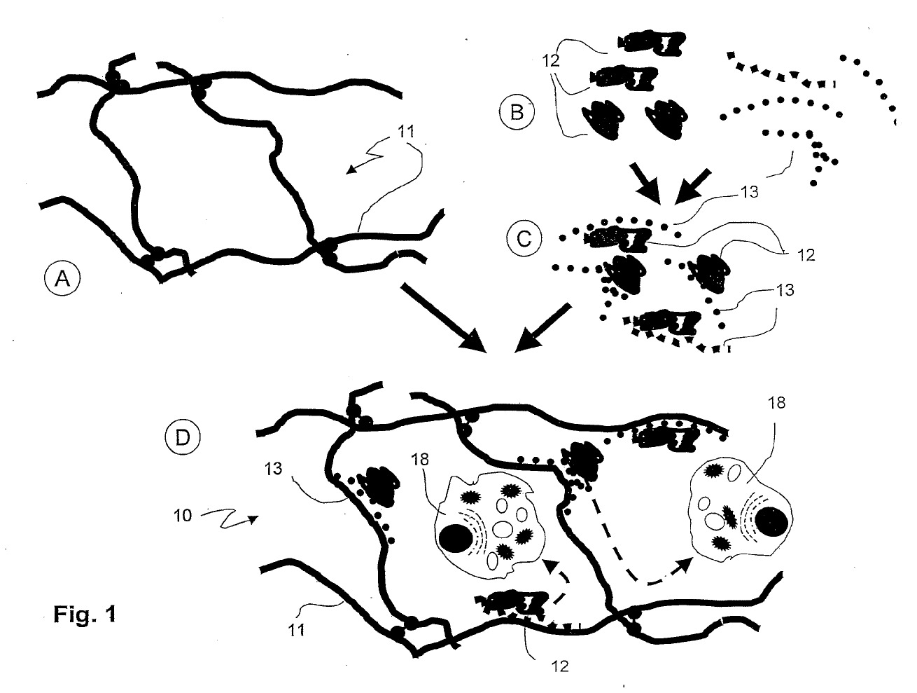

10 in FIG. 1 designates overall a composite biomaterial which is modularly constructed from a support structure 11, a matrix structure 13 and active ingredients 12. In (B) the active ingredients 12 and the matrix structure 13 are provided and in (C) mixed, thus generating by physicochemical interactions matrix-active ingredient complexes which are subsequently combined (D) with the three-dimensional support structure 11 depicted and provided in (A).

The active ingredients 12 may in this case be for example growth factors which are released from the composite biomaterial 10. The active ingredients 12 then come into contact with cells 18 which, on implantation into human or animal tissue, either migrate into the composite biomaterial or surround it and thus come into contact with the released active ingredients/growth factors. On the other side, the cells 18 can, in the case of cell-colonized implants or on use of the composite biomaterial 10 as three-dimensional matrix for cell culturing, also be simply seeded onto the composite biomaterial 10, thus coming into contact once again with the active ingredients to be released.

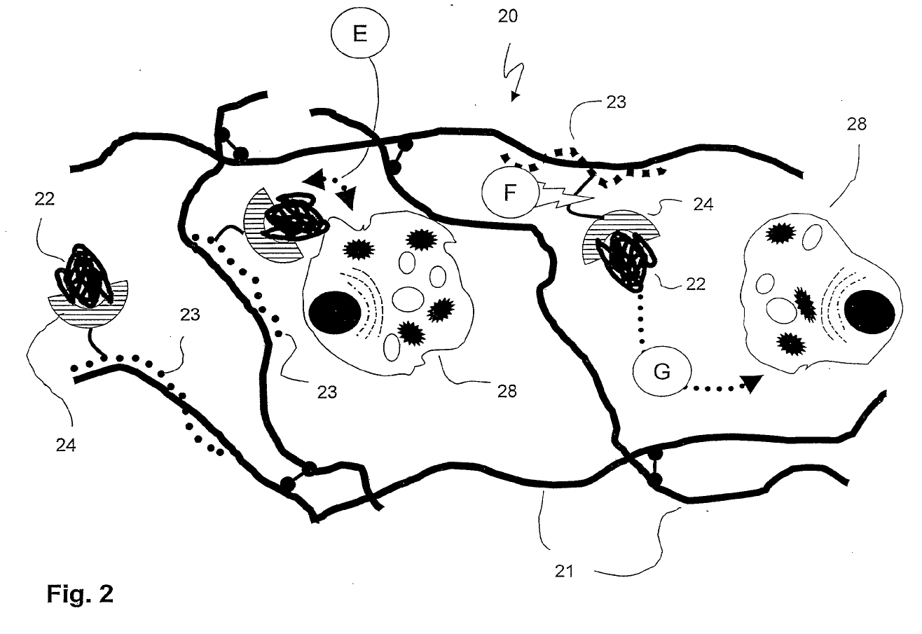

FIG. 2 shows a further embodiment of a composite biomaterial 20 of the invention in which active ingredients 22 are introduced into a three-dimensional support structure 21 via binder molecules 24, for example peptides, which are covalently coupled to matrix components 23. The active ingredients 22 are released over time and/or under physiological conditions (F) and then act on the cells 28 which surround the composite biomaterial or which have migrated into the latter or have been seeded on the latter. Alternatively, the active ingredients 22 bound to binder molecules 24 can also, without previous release, influence (E) cells in the direct vicinity or diffuse away from the binder molecules and interact with cells 28 (G).

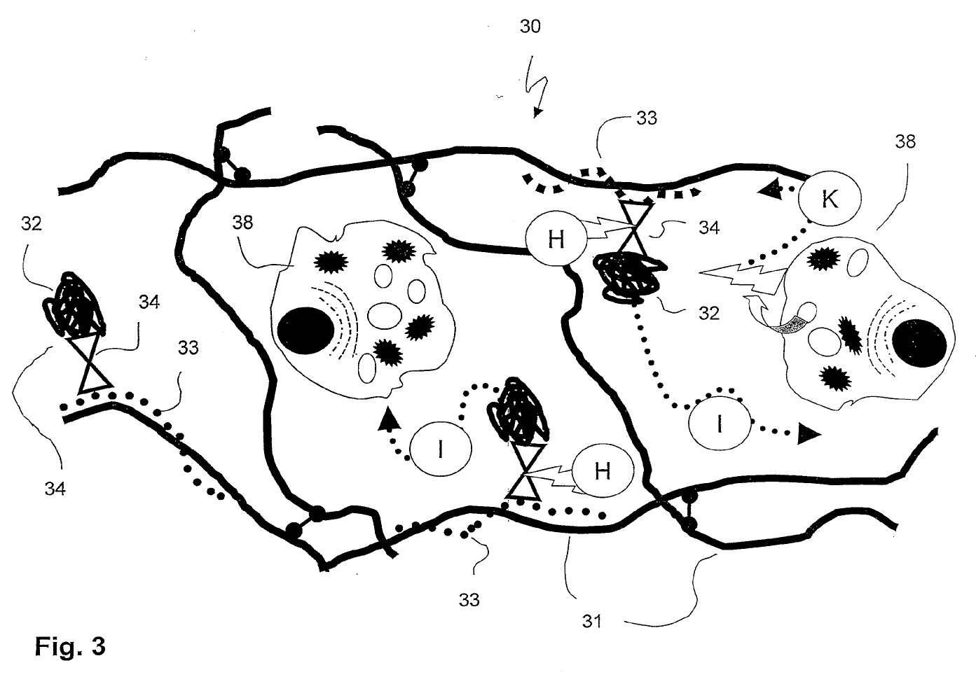

FIG. 3 shows at 30 overall yet a further embodiment of a composite biomaterial of the invention. In this case, the active ingredients 32, for example growth factors, are covalently coupled to a matrix structure 33 via enzyme-cleavable linker molecules 34 which are bound to the three-dimensional support structure 31. The active ingredients 32 are released by an enzymatic cleavage of the linker molecules (H) from the matrix structure and the support structure, and are thus able to act on cells 38 (I). The enzymes, for example proteases, are in this case secreted by the cells which surround the biomaterial in vivo or in vitro (K).

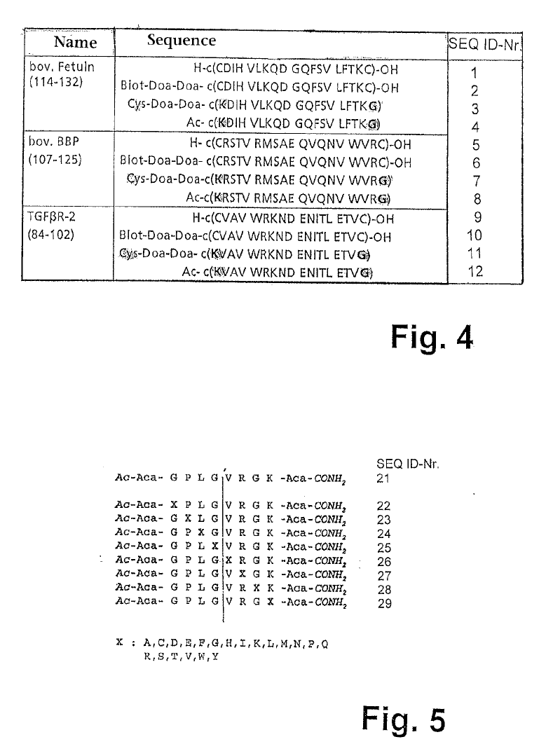

FIG. 4 indicates peptide sequences used for the specific binding of BMP-2 to polymeric supports. The peptides were derived from prior art sequences (Behnam, K., Phillips, M. L., Silva, J. D., Brochmann, E. J., Duarte, M. E., and Murray, S. S. (2005) BMP binding peptide: a BMP-2 enhancing factor deduced from the sequence of native bovine bone morphogenetic protein/non-collagenous protein. J Orthop Res 23, 175-180). Cyclic peptides were prepared by forming disulphide bridges between cystein residues (C) (No. 1, 2 of each series) or by forming a peptide linkage between C-terminus and the E-amino group of terminal lysine (K) (No. 3, 4 of each series). The peptides were synthesized as free (No. 1 of each series) or acetylated peptides (No. 4 of each series) with spacers (Doa: 3,6-dioxa-8-aminooctanoic acid) and biotin (No. 2 of each series) or cystein (No. 3 of each series) for attachment to streptavidin or maleimido groups.

FIG. 5 depicts the diagrammatic structure of the peptide libraries with which the pseudo half-lives of the protease cleavage of certain sequence motifs in peptides were determined. X corresponds to the 20 proteinogenic amino acids. The parallel quantification of the cleaved fragments and of the intact peptides took place by HPLC-electrospray mass spectrometry.

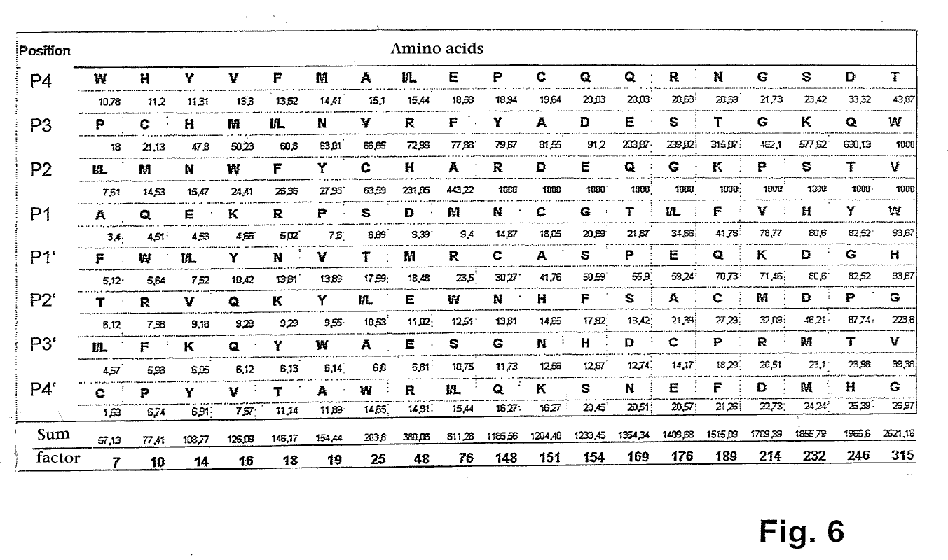

FIG. 6 summarizes the results of the experiments described in FIG. 5. If no cleavage of the peptides was detectable after 24 h, the pseudo half-life for this amino acid at the corresponding position in the sequence was set at 1000. To predict the relative protease lability of a peptide sequence, all pseudo half-lives of the amino acids in the corresponding position to the cleavage site were totalled and divided by the number of positions (in Example 8 for P4-P4′). A smaller factor calculated in this way means faster cleavage of the corresponding peptide sequence by the protease. The values for MMP-7 are shown in the examples.

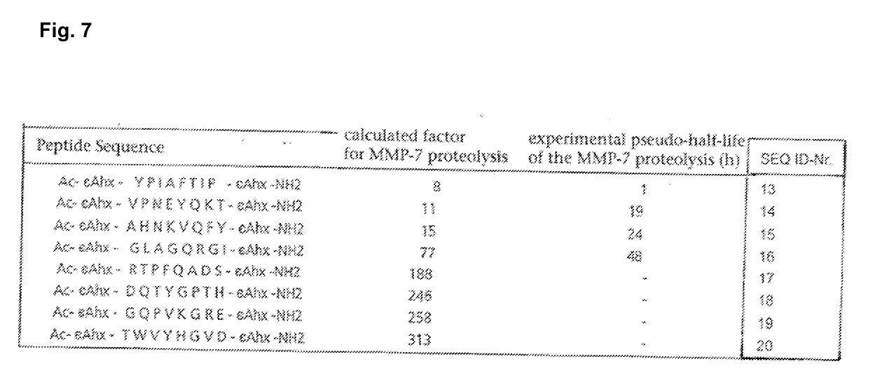

FIG. 7 shows examples of peptide sequences derived from the table in FIG. 6 for MMP-7 substrates. The calculated factors for the proteolysis with MMP-7 are compared with the experimentally determined pseudo half-lives. A good correlation emerges between the prediction and experimentally determined protease lability.

### DESCRIPTION OF PREFERRED EMBODIMENTS

### Examples

### 1.) Nonspecific Binding

A) Introduction of Highly Polymeric Substances into Porous Three-Dimensional Structures of Gelatine Supports.

The experiments were carried out with newly developed gelatine supports (produced in accordance with WO 2005 111 121 A2). The supports were hydrophilized by a single plasma treatment with oxygen. Water-soluble polymers (matrix) such as, for example, short-chain gelatine fractions and active ingredients such as BMP-2 were introduced into the hydrophilized supports within a few seconds into the support, and were uniformly distributed therein. The homogeneous distribution of the polymers taken up in the support structure was testable with fluorescence-labelled (Cy3)-gelatine. The homogeneity of the distribution and the attachment of the high molecular weight matrix structure polymers in the support was demonstrated thereby.

### B) Kinetics of Binding of BMP-2 and Various Types of Gelatine

Surface plasmon resonance analyses with immobilized BMP-2 and various dissolved gelatine samples showed differences in the binding of the different gelatines to the growth factor. Those employed and tested were PS gelatine (type A), Sol-LDA gelatine (type A), LB gelatine (type B) and Sol-D gelatine (type B) (Gelita A G, Eberbach, D)):

| Name | Type | IP | Gel str. |
|---|---|---|---|
| PS | A | about 9 | 300 g bloom |
| Sol LDA | A | about 9 | 0 |
| LB | B | about 5 | 315 g bloom |
| Sol D | B | about 5 | 0 |

(IP = isoelectric point; A = acidic, B = basic hydrolysis; Gel str. = achievable gel strengths)

It emerged that the binding of highly polymeric gelatine was stronger than the binding of low molecular weight gelatine. It was further possible to show that basic gelatine (IP 9, PS, Sol LDA) binds more strongly than acidic gelatine (IP 5, LB, Sol D). BSA (bovine serum albumin) was employed as control.

C) Gelatine Supports with Growth Factors

In accordance with A), gelatine supports were doped with the growth factor bone morphogenetic protein 2 (BMP-2). The supports doped in this way were stored in cell culture medium under culturing conditions for various times. Subsequently, the supernatant of the culture medium was removed and a cell suspension with pluripotent mouse fibroblasts (MC3T3-E1) was added, which fibroblasts differentiate to osteoblasts after activation with BMP-2, and express alkaline phosphatase whose activity can be used as a measure of the stimulation by the growth factor.

The results obtained in these experiments show that the growth factor was active for up to 28 days in the gelatine support.

### 2. Specific Binding

Behnan et al. (J. Orthop. Res. (2005) 23:175-180) disclose BMP-2-binding peptide sequences. These cyclic peptides were modified with linkers by which directed covalent chemical attachment to polymers is possible. It was possible to show the specific binding of BMP-2 to these peptides in accordance with the reference cited above by surface plasmon resonance spectroscopy (SPR, Biacore).

### A) Preparation of Cyclic BMP-2 Binder Peptides

The cyclopeptides mentioned in the literature were synthesized and additionally modified with a linker (biotin, cystein). FIG. 4 shows a tabular overview of the peptides prepared. The peptides were in this case cyclised not only by intramolecular disulphide bridges but also by a peptide linkage between C-terminal glycine and the E-amino group in the N-terminal lysine. It is thus possible to link the cyclopeptide via a spacer and an N-terminal free cystein as thioether covalently to a maleimido-modified matrix structure. The peptides cyclised by disulphide bridges could be linked via a spacer and biotin to streptavidin surfaces. It was possible with these model systems to carry out experiments on BMP-2 binding and release.

### B) Specific Interaction Between Immobilized Cyclopeptides and the Growth Factor BMP-2

SPR analyses were carried out to determine the specific interaction between the cyclopeptides shown in FIG. 4 and BMP-2. For this purpose, BMP-2 was covalently immobilized on a Biacore chip, and kinetics of binding were carried out with solutions of the cyclopeptides in various concentrations. The results obtained thereby show a marked concentration-dependent binding and release of the peptide from the immobilized growth factor.

These results have shown further that the biological activity of BMP-2 is increased by adding cyclic peptides. For this purpose, experiments were carried out with pluripotent mouse fibroblasts (MC3T3-E1) and peptide-bound BMP-2 which differentiate after activation with BMP-2 to osteoblasts and express alkaline phosphatase (ALP). The ALP activity was used as a measure of the stimulation by the growth factor. The binder peptide from FIG. 4 derived from fetuin was employed in the example in this case.

To carry out the experiments, 500 μl of MC3T3 cell suspension were pipetted into each well in a 24-well plate, the cells were subsequently adhered for 4 hours and then incubated with various mixtures (1 ml/well) and tested three times. The ALP activity was measured after 72 hours. The mixtures used were:

Mixture A: BMP-2+binder peptide
0.15 μg of BMP-2/ml of medium (final concentration in the well: 0.1 μg of BMP-2/ml of medium)
3.0 μg of peptide/ml of medium (final concentration in the well: 2.0 μg of peptide/ml of medium)
105 μl of peptide stock solution+5.25 μl of BMP-2 stock solution
Incubation at room temperature for 15 min 3390 μl of MC3T3-E1 complete medium were added to BMP-2/peptide.

Mixture B: Pure BMP-2
0.15 μg of BMP-2/ml of medium (final concentration in the well: 0.1 μg of BMP-2/ml of medium)
105 μl of PBS (pH 5.6)+5.25 μl of BMP-2 stock solution
Incubation at room temperature for 15 min 3390 μl of MC3T3-E1 complete medium were added to BMP-2/peptide.

Mixture C: Pure peptide
3.0 μg of peptide/ml of medium (final concentration in the well: 2.0 μg of peptide/ml of medium)
105 μl of peptide stock solution+5.25 μl of PBS (pH 5.6)
Incubation at room temperature for 15 min 3390 μl of MC3T3-E1 complete medium were added to BMP-2/peptide.

Mixture D: Pure C3T3-E1 complete medium (control without BMP-2 peptide)
3.5 ml of MC3T3-E1 complete medium

Subsequently, 1 ml of medium with BMP-2 was added to all the mixtures, and the mixtures were incubated as mentioned for 72 hours. The ALP activity was then measured. It emerged that the ALP level was higher on incubation of the cells with BMP-2 and peptide than on incubation with BMP-2 alone.

### 3.) Covalent Linkage Via Protease-Labile Linker Peptides

Peptide sequences which can be degraded at different rates by matrix metalloproteases (MMP) were investigated and identified in preliminary experiments.

A) Identification of Maximally Specific MMP Peptide Substrates
Peptide libraries with in each case one randomised amino acid position (19 amino acids) were derived from known MMP peptide substrates with moderate kinetics of proteolysis (see FIG. 5) and were synthesized. Replacement of in each case one amino acid by a mixture of the 20 proteinogenic amino acids in a substrate with a pseudo half-life of 169 in the assay produced a peptide library which was incubated with MMP 7 for different times in each case. The resulting proteolysis fragments were then detected semiquantitatively by HPLC-ESI-MS (high performance liquid chromatography-mass spectrometry with electrospray ionisation).

An increment was calculated from the analyses of the digestions for each amino acid position relative to the enzyme cleavage site and for each amino acid in this position (see FIG. 6). These increments can be used to predict the relative cleavage rates of any different octapeptide sequences.

B) Prediction of the Relative Rate of Proteolysis of Octamer Peptides by MMP-7
Individual peptide substrates differing in expected half-life for the proteolysis by MMP-7 were derived from the increment data (see FIG. 6). These peptides were incubated in individual assays with MMP-7 for different times, and the rate of hydrolysis was analysed by HPLC-ESI-MS in order to determine the experimental pseudo half-lives. There was found to be a good correlation between predicted and pseudo half-life and experimentally determined value (see FIG. 7). It is possible thereby to predict the relative rates of cleavage of octamer peptides on proteolysis with MMP-7 from the increment table (see FIG. 6).

C) Release of a Low Molecular Weight Substrate by MMP-7 Proteolysis
MMP-7-cleavable peptide substrates were covalently coupled N-terminally to BSA (bovine serum albumin) and had a biotin label at the C terminus. The modified BSA molecule was covalently coupled to Luminex XMap™ beads (5 μm diameter) which were incubated with MMP-7 for various times. After the end of the incubation time, the amount of biotin per bead was determined by streptavidin fluorescence on the remaining biotin residues. The streptavidin fluorescence per bead was quantitatively determined using a FACS-analogous system (Luminex 100 System). It was possible to show the release of biotin by MMP-7 activity with various peptide substrates.

It emerged from this that the elimination of the C-terminal, biotinylated peptide fragment after incubation with MMP-7 for various times led to a time-dependent decrease in the biotinylation on the bead surface.

What is claimed is:

1. Composite biomaterial with local, controlled release of active ingredients, comprising a three-dimensional polymer-based support structure, and a polymer-based matrix structure which is introduced into the three-dimensional support structure and homogenously distributed in the support structure, wherein active ingredients are incorporated into the composite biomaterial via the matrix structure.

2. Composite biomaterial according to claim 1, wherein both the support structure and the matrix structure comprise collagenous material or fragments thereof.

3. Composite biomaterial according to claim 1, wherein the active ingredient is selected from at least one of the groups of low molecular weight active ingredients, in particular anti-inflammatory agents, enzyme inhibitors, antibiotics, hormones; growth factors, in particular bone morphogenetic proteins, in particular BMP-2, -7; chemokines; or mixtures thereof.

4. Composite biomaterial according to claim 1, wherein the active ingredient is incorporated into the biomaterial by a nonspecific binding to the matrix structure.

5. Composite biomaterial according to claim 1, wherein the active ingredient is incorporated into the biomaterial via peptides which bind the active ingredient specifically or nonspecifically to the matrix structure.

6. Composite biomaterial according to claim 1, wherein the active ingredient is incorporated into the biomaterial via peptides which bind the active ingredient specifically or nonspecifically to the matrix structure and wherein the peptides binding the active ingredient are bound via a linker to the matrix structure.

7. Composite biomaterial according to claim 1, wherein the active ingredient is incorporated into the biomaterial via peptides which bind the active ingredient specifically or nonspecifically to the matrix structure and, wherein the peptides binding the active ingredient are bound via a linker to the matrix structure, and wherein the attachment of the linker takes place by reactions of the reactants thiol/maleimide, or N-hydroxylamine/aldehyde or amine-aldehyde.

8. Composite biomaterial according to claim 1, wherein the active ingredient is incorporated into the biomaterial via peptides which bind the active ingredient specifically to the matrix structure and wherein the peptide which binds the active ingredient is derived from a protein which is selected from bovine fetuin, bovine BBP or TGFβR-2.

9. Composite biomaterial according to claim 1, wherein the active ingredient is incorporated into the biomaterial via peptides which bind the active ingredient specifically to the matrix structure and wherein the peptide which specifically binds the active ingredient has a sequence which is selected from one of the SEQ ID No. 1 to 12 from FIG. 4 or the appended sequence listing.

10. Composite biomaterial according to claim 1, wherein the active ingredient is incorporated into the biomaterial via peptides which bind the active ingredient specifically to the matrix structure and wherein the active ingredient is growth factor BMP-2 (bone morphogenic protein 2) which is specifically bound via cyclopeptides specific for BMP-2.

11. Composite biomaterial according to claim 1, wherein the active ingredients are covalently linked to the matrix structure via peptides which are enzymatically cleavable by a matrix metalloprotease (MMP), which is selected from MMP-7, MMP-1, MMP-3, MMP-8, MMP-9, MMP-10, or MMP-13.

12. Composite biomaterial according to claims 1, wherein the active ingredients are covalently linked to the matrix structure via peptides which are enzymatically cleavable by a matrix metalloprotease (MMP), and wherein the peptide covalently linked to the matrix structure has a sequence which is selected from one of the SEQ ID No. 13 to 20 from FIG. 7 or the appended sequence listing.

13. Process for producing a composite biomaterial comprising a three-dimensional polymer-based support structure, and a polymer-based matrix structure, comprising the following consecutive steps:
a) separate provision of i) polymer-based support material and ii) polymer-based matrix material;
b) hydrophilization of the support material; and
c) introduction of the matrix material provided in step a) into the hydrophilized support material from step b) to produce a composite biomaterial with a support structure and a matrix structure.

14. Process according claim 13, wherein a matrix material to which active ingredients are bound is provided in step a) to produce a composite biomaterial with a support structure and an active ingredient-doped matrix structure.

15. Method for treating degenerated or diseased tissue in a patient in need thereof, comprising the step of administering or implanting a composite biomaterial to said patient, said composite biomaterial having a local, controlled release of active ingredients, and comprising a three-dimensional polymer-based support structure, and a polymer-based matrix structure which is introduced into the three-dimensional support structure and homogenously distributed in the support structure, wherein active ingredients are incorporated into the composite biomaterial via the matrix structure.

16. Method for treating cancer in a patient suffering from cancer, comprising the step of administering or implanting a composite biomaterial having a local, controlled release of active ingredients, and comprising a three-dimensional polymer-based support structure, and a polymer-based matrix structure which is introduced into the three-dimensional support structure and homogenously distributed in the support structure, wherein active ingredients are incorporated into the composite biomaterial via the matrix structure to said patient, and of the local delivering of chemotherapeutics released from said composite biomaterial in said patient.

17. Method for cultivating cells in vitro, comprising the steps of providing a composite biomaterial having a local, controlled release of active ingredients, and comprising a three-dimensional polymer-based support structure, and a polymer-based matrix structure which is introduced into the three-dimensional support structure and homogenously distributed in the support structure, wherein active ingredients are incorporated into the composite biomaterial via the matrix structure, seeding said composite biomaterial with at least one cell or cell population, and cultivating the cells in the composite biomaterial under conditions that promote growth of said cells.


SEQUENCE LISTING (29 sequences)

SEQ ID NO: 1 - protein, 19 aa, Artificial Sequence
  Linker
CDIHVLKQDGQFSVLFTKC

SEQ ID NO: 2 - protein, 19 aa, Artificial Sequence
  Linker
CDIHVLKQDGQFSVLFTKC

SEQ ID NO: 3 - protein, 19 aa, Artificial Sequence
  Linker
KDIHVLKQDGQFSVLFTKG

SEQ ID NO: 4 - protein, 19 aa, Artificial Sequence
  Linker
KDIHVLKQDGQFSVLFTKG

SEQ ID NO: 5 - protein, 19 aa, Artificial Sequence
  Linker
CRSTVRMSAEQVQNVWVRC

SEQ ID NO: 6 - protein, 19 aa, Artificial Sequence
  Linker
CRSTVRMSAEQVQNVWVRC

SEQ ID NO: 7 - protein, 19 aa, Artificial Sequence
  Linker
KRSTVRMSAEQVQNVWVRG

SEQ ID NO: 8 - protein, 19 aa, Artificial Sequence
  Linker
KRSTVRMSAEQVQNVWVRG

SEQ ID NO: 9 - protein, 18 aa, Artificial Sequence
  Linker
CVAVWRKNDENITLETVC

SEQ ID NO: 10 - protein, 18 aa, Artificial Sequence
  Linker
CVAVWRKNDENITLETVC

SEQ ID NO: 11 - protein, 18 aa, Artificial Sequence
  Linker
KVAVWRKNDENITLETVG

SEQ ID NO: 12 - protein, 18 aa, Artificial Sequence
  Linker
KVAVWRKNDENITLETVG

SEQ ID NO: 13 - protein, 8 aa, Artificial Sequence
  Linker
YPIAFTIP

SEQ ID NO: 14 - protein, 8 aa, Artificial Sequence
  Linker
VPNEYQKT

SEQ ID NO: 15 - protein, 8 aa, Artificial Sequence
  Linker
AHNKVQFY

SEQ ID NO: 16 - protein, 8 aa, Artificial Sequence
  Linker
GLAGQRGI

SEQ ID NO: 17 - protein, 8 aa, Artificial Sequence
  Linker
RTPFQADS

SEQ ID NO: 18 - protein, 8 aa, Artificial Sequence
  Linker
DQTYGPTH

SEQ ID NO: 19 - protein, 8 aa, Artificial Sequence
  Linker
GQPVKGRE

SEQ ID NO: 20 - protein, 8 aa, Artificial Sequence
  Linker
TWVYHGVD

SEQ ID NO: 21 - protein, 8 aa, Artificial Sequence
  Linker
GPLGVRGK

SEQ ID NO: 22 - protein, 8 aa, Artificial Sequence
  Linker
XPLGVRGK

SEQ ID NO: 23 - protein, 8 aa, Artificial Sequence
  Linker
GXLGVRGK

SEQ ID NO: 24 - protein, 8 aa, Artificial Sequence
  Linker
GPXGVRGK

SEQ ID NO: 25 - protein, 8 aa, Artificial Sequence
  Linker
GPLXVRGK

SEQ ID NO: 26 - protein, 8 aa, Artificial Sequence
  Linker
GPLGXRGK

SEQ ID NO: 27 - protein, 8 aa, Artificial Sequence
  Linker
GPLGVXGK

SEQ ID NO: 28 - protein, 8 aa, Artificial Sequence
  Linker
GPLGVRXK

SEQ ID NO: 29 - protein, 8 aa, Artificial Sequence
  Linker
GPLGVRGX